(19) United States
(12) Patent Application Publication
(10) Pub. No.: US 2022/0062441 A1
(43) Pub. Date: Mar. 3, 2022

(54) METHODS FOR IMAGING AND TREATING DAMAGED CARTILAGE

(71) Applicant: The Brigham and Women`s Hospital, Inc., Boston, MA (US)

(72) Inventors: Shuichi Mizuno, Brookline, MA (US); Kenichi Maeda, Boston, MA (US)

(21) Appl. No.: 17/421,320

(22) PCT Filed: Jan. 6, 2020
(86) PCT No.: PCT/US2020/012368

§ 371 Date: Jul. 7, 2021

Related U.S. Application Data
(60) Provisional application No. 62/789,350, filed on Jan. 7, 2019.

(51) Int. Cl. A61K 49/00; A61K 38/38; A61B 1/04; A61K 47/62

(52) U.S. Cl. CPC A61K 49/0056; A61K 47/62; A61B 1/043; A61K 38/385

(57) ABSTRACT

Methods of imaging and treating damaged cartilage, e.g., damaged cartilage surfaces, using cationic albumin Described herein is the development of contrast agents comprising cationic albumin (C-albumin) and a cyanine dye to illuminate damaged articular cartilage, and methods of treatment using the C-albumin.

### CLAIM OF PRIORITY

This application claims the benefit of U.S. Patent Application Ser. No. 62/789,350, filed on Jan. 7, 2019. The entire contents of the foregoing are hereby incorporated by reference.

### TECHNICAL FIELD

Provided herein are methods of imaging and treating damaged cartilage, e.g., damaged cartilage surfaces, using cationic albumin.

### BACKGROUND

Articular cartilage provides a smooth, lubricating surface in joints facilitating easy movement even under load. When damaged, e.g., after injury, accidents, or wear, articular cartilage has little capacity for healing and without treatment can lead to painful friction between the bones of the joints and loss of function, including long term disease. See, e.g., Fox et al., Sports Health. 2009 November; 1(6): 461-468. For example, joint injuries (such as meniscal and ligament tears, and cartilage defects) in young adults significantly raise the risk of developing progressive early osteoarthritis (OA). Recently, post traumatic osteoarthritis is increasing.

### SUMMARY

Described herein is the development of contrast agents comprising cationic albumin (C-albumin) and a cyanine dye to illuminate damaged articular cartilage, and methods of treatment using the C-albumin. As shown herein, these contrast agents comprising C-albumin-ICG can be used to illuminate damaged surfaces that are invisible with regular arthroscopy procedure. This agent reduces the likelihood that damage will go undetected. Since OA has an early onset after knee injury, this procedure will be useful to notice the damage. In addition, C-albumin can be used to promote resurfacing of the damaged articular cartilage.

Thus, provided herein are methods for treating an area of damage to a surface of articular cartilage, comprising contacting the damaged surface with an effective amount of cationized-albumin (C-albumin). Also provided herein is C-albumin for use in a method of treating an area of damage to a surface of articular cartilage.

In some embodiments, the C-albumin has a net charge of 23±3.

In some embodiments, the C-albumin is in a composition further comprising a hydrogel that increases viscosity. In some embodiments, the hydrogel comprises one or more of collagen, gelatin, alginate, hyaluronic acid, heparin, chondroitin sulfate, chitosan, poly(ethylene glycol) (PEG), or poly(vinyl alcohol).

Also provided herein are methods for identifying an area of damage to a surface of articular cartilage, comprising contacting the surface with a sufficient amount of cationized-albumin (C-albumin) conjugated to a near-infrared (NIR) imaging moiety;

irradiating the surface with near-infrared light from a source; detecting an area of fluorescence emitted by the NIR imaging moiety, and identifying the area of fluorescence as a damaged area.

In some embodiments, the near-infrared imaging moiety is indocyanine green (ICG), and the method comprises irradiating the surface with light between 600 nm and 900 nm, and detecting emitted fluorescence between 750 nm and 950 nm.

In some embodiments, the methods include using an endoscope that emits near-infrared light and detects fluorescence emitted by the NIR imaging moiety.

In some embodiments, once the damaged area is identified, the methods further include administering a treatment as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-O. Cell proliferation by synovium cells, meniscal cells, and chondrocytes with pulse treatment of C-albumin, protamine, and poly-1-lysine, BSA, and chondroitin sulfate.

FIGS. 2A-I. Cell proliferation by synovium cells, meniscal cells, and chondrocytes with pulse treatment of C-albumin, protamine, and poly-1-lysine.

FIG. 3. Adhered human synovium cells onto middle layer of bovine articular cartilage after 1 day of culture. The cells were labeled with fluorescent cell tracker (PKH26). The tissues were incubated in test agents dissolved in F12 medium without serum for 3 hours at 37° C. in 5% CO₂. Top Row, Phosphate Buffer Saline Control. Middle Row, Human Albumin Control. Bottom Row, Cationic-Human Albumin.

FIG. 4. Photographs of damaged articular cartilage illuminated with C-albumin-ICG.

FIG. 5. Effects of human cationic albumin on cellular characteristics of proliferation of human articular chondrocytes.

### DETAILED DESCRIPTION

Articular cartilage has abundant sulfated proteoglycan, which present a hydrophobic nature and little cell adhesion molecules. Thus, cells from adjacent tissues e.g., synovium membrane and meniscus, do not typically migrate to articular cartilage.

Joint injuries in young adults significantly raise the risk of developing progressive early osteoarthritis (OA). These injuries primarily include meniscal and ligament tears, and cartilage defects. During treatments for these injuries, early surface degeneration in articular cartilage has a risk to be overlooked with arthroscopy because the damage is invisible. To solve this diagnostic problem, we developed a novel near infrared fluorescent contrast agent using indocyanine green (ICG) combined with cationic serum albumin (C-albumin) to pick up surface damage.

The present experiments showed that C-albumin synthesized with human and bovine serum albumin were both useful in imaging damage. In addition, we found that the C-albumin had a potential to stimulate cell proliferation by synovium derived cells in vitro.

Thus the C-albumin can be used for picking up damaged surface and, at the same time, for promoting repair of the damaged surface of articular cartilage. We demonstrated the ability of C-albumin to promote cell proliferation using synovium membrane-derived cell, meniscal cells, and articular chondrocytes in vitro.

Furthermore, we compared the capability of cell proliferation by C-albumin with other cationic molecules: protamine, poly-1-lysine; anionic molecule: chondroitin sulfate; and neutralized molecule: pure bovine serum albumin. We exposed the aforementioned cells to C-albumin, protamine, and poly-1-lysine for 3 hours followed by replacing with growth medium. C-albumin had the most potential stimulatory effects with synovium cells, meniscal cells, and chondrocytes as compared to the other cationic molecules, protamine and poly-1-lysine. Without wishing to be bound by theory, it is hypothesized that this effect is not only a charge specific event; it is believed that the cationic molecules neutralized anionic cartilage matrix, which allowed cell adhesion followed by proliferation. In addition, the capability of cell proliferation was seen at a wide range of dosage. Thus, C-albumin likely has a good safety profile.

The C-albumin-ICG contrast agent infiltrated and retained in the underneath tissue (middle layer) of the surface. On the other hand, it did not infiltrate from the intact surface. Thus, C-albumin-ICG can be used to image and detect damaged surfaces in articular cartilage, which is invisible with regular visual light source and arthroscopy settings, as well as promoting covering damaged articular surface.

### C-Albumin and C-Albumin Imaging Reagents

At pH 7.4, the net charge of serum albumin is −19±2 (see, e.g., Akdogan et al., Phys. Chem. Chem. Phys., 2016, 18, 22531-22539; and US20080308744); in the present methods and compositions, the albumin is cationized, optionally to have a net charge of +20±6 or +23±3 (e.g., as described in Akdogan et al., Phys. Chem. Chem. Phys., 2016, 18, 22531-22539), or pI=8, or about 8 (e.g., ±10%), by isoelectrofocus (IEF). In general, the species of albumin used should match that of the species of the subject to be treated, e.g., human albumin should be used to treat human subjects. The following table provides exemplary sequences for albumin:

| Species | NCBI RefSeq Acc. No. |
|---|---|
| H. sapiens | NP_000468.1 |
| P. troglodytes | XP_517233.3 |
| M. mulatta | XP_001103956.1 |
| C. lupus | NP_001003026.1 |
| B. taurus | NP_851335.1 |
| M. musculus | NP_033784.2 |
| R. norvegicus | NP_599153.2 |
| G. gallus | NP_990592.1 |
| F. cattus | NP_001009961.1 |
| E. caballus | NP_001075972.1 |

Methods for making purified albumin are known in the art, and can include purification from natural sources or recombinant production, e.g., in yeast; see, e.g., WO1996037515A1; U.S. Pat. No. 7,531,631; US20120149873; WO2013006675; WO2018065491; U.S. Pat. No. 5,728,553; US20050054051; JP2004536081; WO2016028790A1; and WO2010092135.

In some embodiments, the compositions and methods described herein include C-albumin conjugated to an imaging moiety, preferably a near-infrared imaging moiety. Preferred near-infrared imaging moieties include indocyanine green (ICG). Methods of making such reagents are known in the art. See, e.g., U.S. Pat. No. 9,493,545; US20070059244; U.S. Pat. No. 8,227,621; and US20080308744 (which describes conjugation by non-specific adsorption of serum albumin with ICG).

### Methods of Diagnosis

The present disclosure provides methods for diagnosing and identifying damage to articular cartilage, e.g., damage to the surface of articular cartilage. The methods include administering or applying the C-albumin imaging reagents described herein; optionally waiting for a period of time sufficient for the reagents to distribute into the underlying tissue through any damaged areas in the surface, then washing the reagents out; and using an appropriate imaging method to detect the presence of the imaging reagent in the underlying cartilage tissue. Since undamaged cartilage surface doesn't allow penetration of the imaging reagent into the underlying tissue (e.g., middle (transitional) zone and/or deep zone), the presence of the imaging reagent indicates the adjacent surface is damaged. For example, the methods can proceed as follows. During arthroscopy for diagnosis and/or treatment, the joint space is flushed with saline, then the C-albumin-ICG is injected. After a sufficient time, e.g., 10-30 minutes, e.g., 15 minutes, the unbound C-albumin-ICG is washed out with saline, and then the cartilage surface is observed with near infrared arthroscopy. Before closing the wound or access ports, additional C-albumin can be injected into the joint. Furthermore, the C-albumin can be injected into the joint post operatively, or in the absence of imaging methods.

Imaging methods that can be used in the method described herein include near-infrared arthroscopy, e.g., as described in U.S. Pat. No. 9,877,654; Sarin et al., Scientific Reports volume 8, Article number: 13409 (2018) (near-infrared spectroscopy); WO2012016224; US20140243680; US20100256504; and others.

### Methods of Treatment

Further provided herein are methods for treating damaged surfaces of articular cartilage. The methods include administering to the surface a therapeutically effective amount of C-albumin, e.g., of a composition comprising C-albumin. A “therapeutically effective amount” is an amount that improves one or more symptoms associated with the damage, e.g., reduces damage to the surface of the cartilage, improves joint mobility, and/or reduces pain, soreness, or stiffness of the joint.

### Pharmaceutical Compositions and Methods of Administration

The methods described herein include the use of pharmaceutical compositions comprising C-albumin or C-albumin-ICG as an active ingredient.

Pharmaceutical compositions typically include a pharmaceutically acceptable carrier. As used herein the language “pharmaceutically acceptable carrier” can include saline, solvents, dispersion media, antibacterial and antifungal agents, isotonic and absorption delaying agents, and the like, compatible with pharmaceutical administration. Supplementary active compounds can also be incorporated into the compositions, e.g., Small molecules and growth factors e.g., transforming growth factor-beta.

Pharmaceutical compositions are typically formulated to be compatible with its intended route of administration. In the present case, the compositions are administered by injection.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. The pharmaceutical compositions can be included in a container, pack, or dispenser together with instructions for administration.

The compositions can include one or more carriers that increase viscosity to increase retention time in the joint. Such carriers can include silicone gels and hydrogels, e.g., collagen or gelatin, alginate, hyaluronic acid, heparin, chondroitin sulfate, chitosan, poly(ethylene glycol) (PEG), and poly(vinyl alcohol); see, e.g., U.S. Pat. Nos. 6,960,617; 9,468,683; 9,114,188; WO2002087645; and Liu et al., Bone Research 5:17014 (2017).

### EXAMPLES

The invention is further described in the following examples, which do not limit the scope of the invention described in the claims.

### Materials and Methods

The following materials and methods were used in the examples below.

### Modification of Serum Albumin

C-albumin was prepared from bovine or human serum albumin with anhydrous ethylenediamine, HCl, and 1-ethyl-3-(3-dimethyl-aminopropyl)-carbo-diimidehydrochloride at pH 4.7. The C-albumin solution was filtered and dialyzed with deionized water for 72 hr at 4° C., then lyophilized for the experiments. It was then dissolved in HPLC grade water and purified with ultrafiltration membranes (30 KD followed by 100 KD). The C-albumin between MW 30 and 100 KD was used for further experiments. The concentration and quality of C-albumin were determined by BCA assay and by isolectrophoresis and SDS-PAGE with a 3-8% Tris-acetate gel, respectively.

### Isolation of Chondrocytes, Meniscus Cells and Synovial Cells

We harvested the articular cartilage, meniscus and synovium membrane aseptically from bovine knee purchased from a local slaughterhouse (IACUC was not required). The cartilage, meniscus and synovium were minced with a blade (#22, Bard-Parker, SE Caledonia, Mich.). The minced tissues were digested in 0.15% collagenase type 1 (Worthington, Lakewood, N.J.) dissolved in F-12 Nutrient Mixture (Life Technology, Waltham, Mass.) for 16 hours at 37° C. on a rotator shaker in a thermal incubator. The digest was centrifuged (100×g, 5 min) and supernatant was removed. The cell pellet was rinsed in phosphate buffer saline (PBS, Life Technology) with centrifugation (100×g, 5 min). The cells were resuspended in Dulbecco Minimal Essential Medium (DMEM, Life Technology) containing 10% fetal bovine serum (FBS, Life Technology) and antibiotics. We seeded the cell suspension to cell culture dishes and incubated at 37° C. in an incubator under 5% CO₂.

### Preparation of Test Agents

We added BSA (Sigma-Aldrich, St. Louis, Mo.), C-albumin, protamine (Sigma-Aldrich, St. Louis, Mo.) and chondroitin sulfate (CS, Sigma-Aldrich) to DMEM containing 10% FBS and antibiotics at the following concentrations. The highest concentration of each agent dissolved in the medium was sterilized with a sterile syringe filter (0.45 μm, Millipore). The sterilized solutions were further diluted in DMEM containing 10% FBS and antibiotics to prepare the solutions below (Table 1).

TABLE 1
Test agents

| Agents | Dosage |
|---|---|
| Cationic bovine serum albumin (C- | 0.01, 0.03, 0.1, 0.3, 1.0 (mg/ml) |
| albumin) | |
| Protamine | 0.001, 0.003, 0.01, 0.03, 0.1 |
| Poly-L-lysine | 0.0001, 0.0003, 0.001, 0.003, 0.01 |
| Bovine serum albumin (BSA) | 0.01, 0.03, 0.1, 0.3, 1.0 |
| Chondroitin sulfate from shark (CS) | 0.01, 0.03, 0.1, 0.3, 1.0 |

### Cell Proliferation Assays

### Medium Substitution

We seeded 1 ml of five thousand cells/mL cell suspension into 12-well plates and incubated them for 20 hours at 37° C. in 5% CO₂. After incubation, we rinsed each well with PBS and added the test solutions dissolved in culture medium. The non-treatment control was prepared without a test agent (you previously had a double negative). The plate was incubated for 4 days at 37° C. in 5% CO₂. At day 4, we harvested the cells with 0.1 mL of 0.05% trypsin (Life Technology) and counted the cells. We counted the cells with a trypan blue and a hematocytometer.

### Pulse Exposure

We seeded 1 ml of five thousand cells/mL cell suspension to 12-well plates and incubated them for 20 hours at 37° C. in 5% CO₂. After incubation, we added C-albumin 0.01 mg/ml, protamine 0.03 mg/ml, and poly-1-lysine 0.003 mg/ml to the cells for 3 hr (pulse exposure) and changed the medium followed by incubation for 24, 48, 72 and 96 hr. We counted the cells with a trypan blue and a hematocytometer. We evaluated the cell number of each cell type with treatment of C-albumin and others using a Bonferroni-test. For statistical analysis, we repeated these experiments three times.

### Cell Adhesion and Morphology

To more closely model in vivo behaviour, we seeded human synovium membrane derived cells onto a middle layer of articular cartilage piece harvested from bovine shoulder joint. We trimmed the cartilage piece into 5×5 mm area and removed the surface layer. The pieces were incubated in test agent solution for 4 hr at 37° C. in 5% CO₂ followed by rinse in PBS. The passaged synovium cell suspension was incubated with a cell tracker (PKH26, Sigma-Aldrich) for 5 min. We seeded the labeled 5,000 cells suspended in 25 μl of medium onto the cartilage piece. The cell suspension could stay on the piece with surface tension. If the cell suspension was spread to outside of surface, this piece was removed from evaluation. The cells adhered on the surface of cartilage. These pieces were transferred to new 12-wells plates and continued to be incubated for 24 hr. At 24 hr, we flipped over the piece and placed on a coverslip. We observed the pieces with a fluorescent inverted microscope. To evaluate cellular morphology of human synovium cells, we observed them with a fluorescent dye (PKH 26, Ex: 551 nm, Em: 567 nm) at 24 hr after seeding.

### Detection of Damaged Surface with C-Albumin/ICG Ex Vivo

Albumin-ICG, C-albumin-ICG, and ICG alone (0.156 mg/ml) were compared for specificity and intensity against the damaged bovine articular cartilage using a CCD camera (ORCA-ER) at ex: 760 nm LED, em: 810 nm filter. In order to create wear, full-thickness pieces (2 cm²) of normal articular cartilage were scratched with a flat surface pin with a sand paper (#150, 4 mm in diameter). The cartilage pieces were rinsed with saline, flipped over, and placed on the testing contrast agents (0.5 ml) for 15 min followed by rinsing with saline for evaluation. Only the surface was immersed into the solution due to surface tension. Fluorescent images at the surface were recorded with a CCD camera. After imaging, the cartilage was fixed with 2% paraformaldehyde and embedded in hydroxyethylmethacrylate. Five-μm sections were stained with Toluidin blue-O.

### Formation of Cartilage Organoids

Chondrocytes were isolated enzymatically from discarded articular cartilage underwent total knee arthroplasty. We expanded these chondrocytes in monolayered culture. After one or two passages, we seeded five thousand cells suspended in 150 μl into a round bottom well of 96-wells plates. The chondrocytes gradually formed a spherical organoid ranging 300-500 μm in diameter in 3-5 days.

We removed medium from each well and added 150 μl of new medium containing Human cationic albumin at 0, 0.01, and 0.1 mg/ml. We incubated these organoids for additional 4 days. At 2 and 4 days after adding the cationic albumin, we harvested these organoids for mRNA analysis and extracted total RNA using RNeasy kit (Qiagen). We repeated this experiment four time for gaining statistical power.

### Quantification of Gene Expression

The RNA samples were amplified with a reverse transcriptase (high-capacity cDNA reverse transcription kit, Life Technology) and a mixed gene expression master mix and fluorescent-labeled specific primers (TaqMan®, Life Technology), followed by quantitative-PCR (QuantStudio™, Applied Biosystems, Foster City, Calif.). We chose the TaqMan® Primers: collagen type-1 (COL-1), Hs00164004_m1; integrin subunit alpha V (ITGA-V), Hs00233808_m1; integrin subunit alpha 11 (ITGA11), Hs01012939_m1; proliferating cell nuclear antigen (PCNA), Hs00427214 and glyceraldehyde-3-phosphate dehydrogenase (GAPDH), Hs03929097_g1. We used Expression Suite Software v.1.0.4 to convert from ΔCt to relative quantity (RQ).

### Results

The molecular weight and isoelectric point of C-albumin were 60K-70K and higher than p18, respectively.

### Cell Proliferation Assays

### Medium Supplement

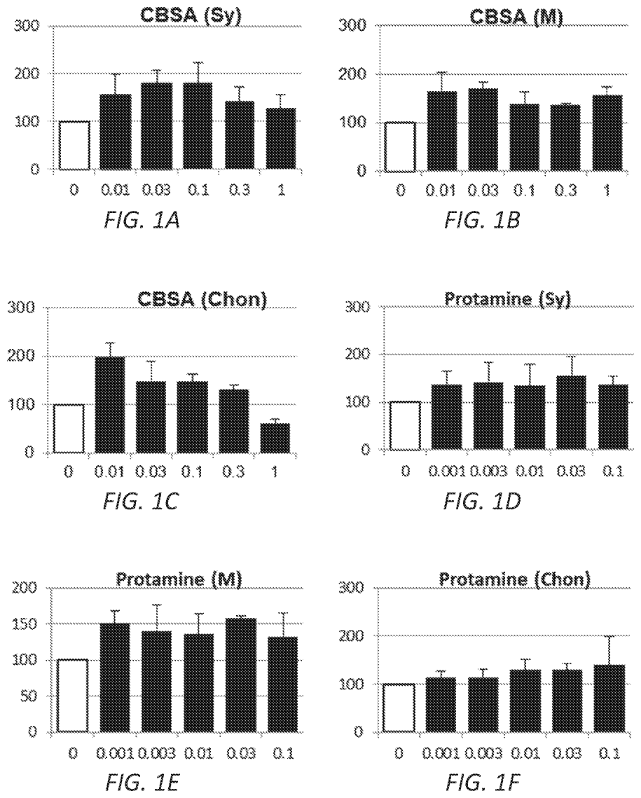

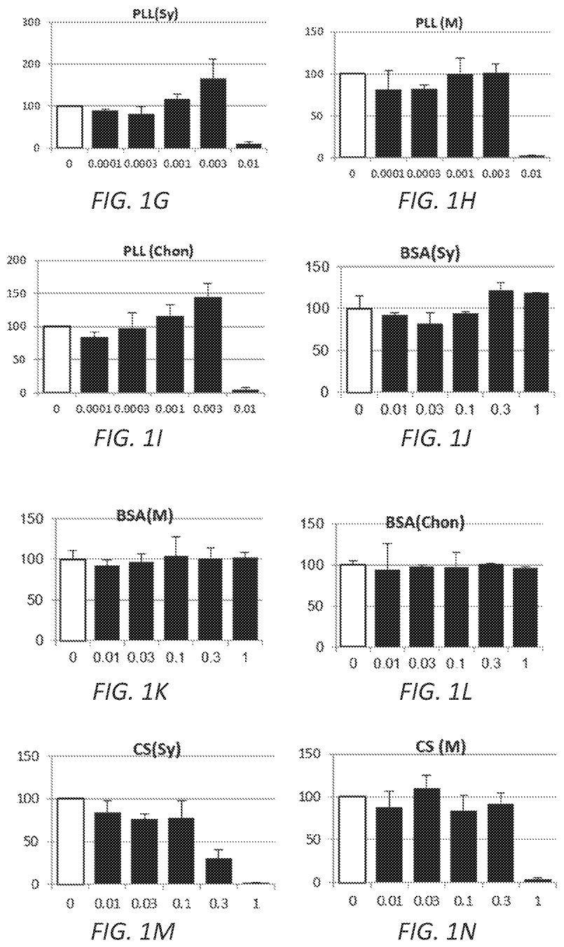

We clarified the effects of C-albumin and other cationic agents on proliferation of various cell types. C-albumin significantly promoted cell proliferation by synovium cells and chondrocytes (0.01-0.1 mg/ml, P<0.05) but not meniscal cells. C-albumin at 1 mg/ml, however, inhibited chondrocyte proliferation (FIGS. 1A, B, C).

Protamine showed trends of high proliferation with synovium cells and meniscal cells at 0.001-0.1 mg/ml, but it was not significant (FIGS. 1D, E, F).

Poly-1-lysine at 0.003 mg/ml showed significant high proliferation by synovium cells, whereas significant inhibition at 0.01 mg/ml (FIGS. 1G, H, I). Poly-1-lysine at 0.01 mg/ml showed significant inhibition of proliferation by all synovium, meniscal cells and chondrocytes.

BSA showed similar proliferation levels at all concentration (FIGS. 1J, K, L).

Chondroitin sulfate showed similar proliferation capability by synovium cells at 0.01-0.1 mg/ml, meniscal cells at 0.01-0.3 mg/ml, and by chondrocytes at 0.01-0.03 mg/ml, respectively, whereas, showed inhibition at higher concentration of each agent (FIG. 1M, N, O).

### Pulse Exposure

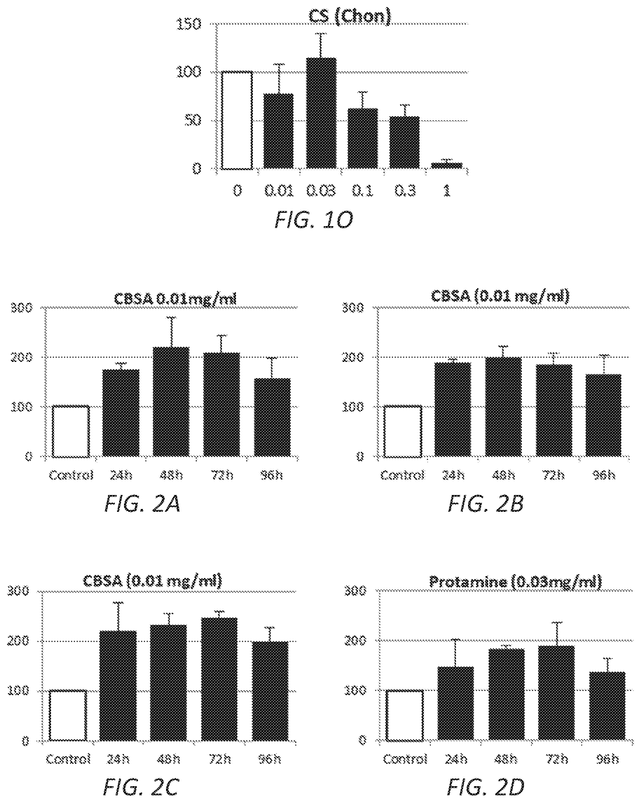

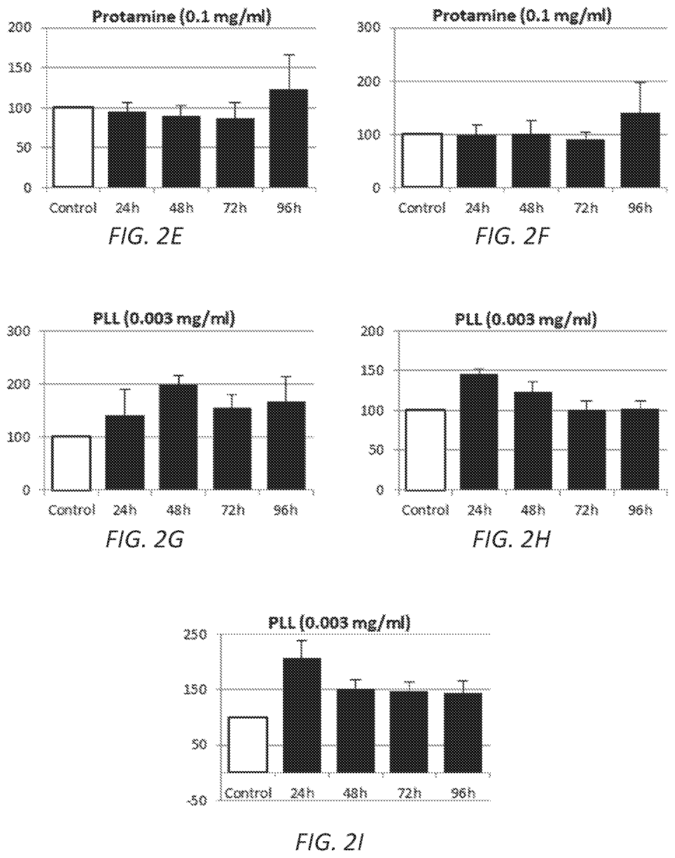

We clarified the effects of pulse exposure of C-albumin and other cationic agents on capability of proliferation by various cell types. C-albumin stimulated significantly cell proliferation by synovium cells and chondrocytes (0.01-0.1 mg/ml, P<0.05) but not meniscal cells (FIGS. 2A, B, C).

Protamine stimulated significant proliferation by synovium cells by 72 hr, whereas declined at 96 hr. Meniscal cells and chondrocytes retained cell numbers by 72 hr and slightly increased at 96 hr (FIGS. 2D, E, F).

Poly-1-lysine stimulated proliferation by synovium cells by 48 hr (FIG. 2G). It also stimulates proliferation by meniscal cells and chondrocytes by 24 hr, whereas declined afterward (FIGS. 2G, H, I).

### Cell Adhesion and Morphology

In preliminary experiments, we examined adherent capability by synovium cells on a surface of articular cartilage that had been scratched with sand paper. We tried to optimize methodology, however, the cells attached to the surface inconsistently. Since the intact surface layer of cartilage was hydrophobic, the cell suspension was not homogeneously spread. In order to expose middle layer of cartilage, we removed the surface layer with a surgical blade completely.

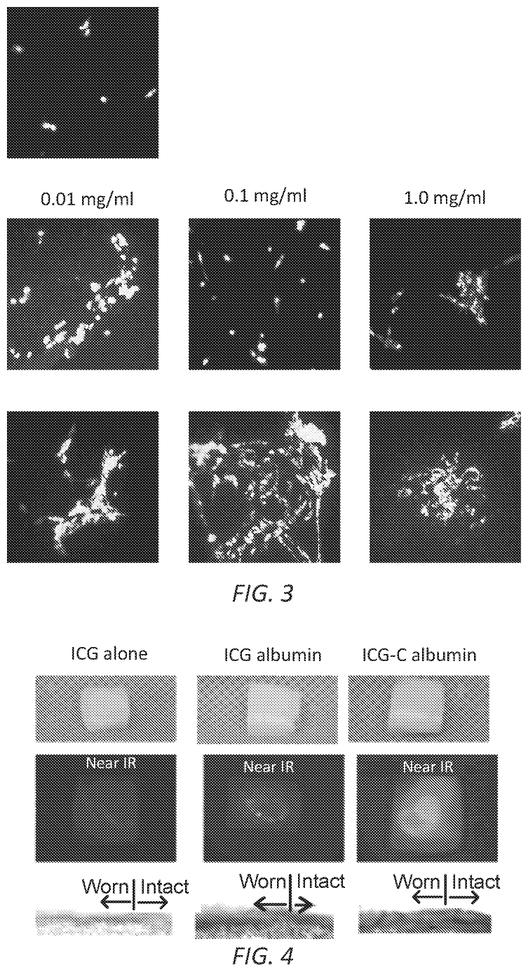

Human synovium cells adhered on the surface of the middle layer of articular cartilage and did not adhere on the surface layer (FIG. 3). The synovium cells elongated on the surface of the middle layer treated with C-albumin (0.1 mg/ml). On the other hand, the cells maintained round shape and fewer cells attached on the non-treated and treated cartilage with human serum albumin (FIG. 3).

### Detection of Damaged Surface with C-Albumin/ICG Ex Vivo

Albumin-ICG, C-albumin-ICG, and ICG alone were compared by imaging for specificity and intensity of damaged and undamaged bovine articular cartilage.

With an optimal concentration (e.g., about 0.156 mg/ml) of C-albumin, albumin, and ICG alone, intact cartilage surface was unstained. This concentration minimized non-specific binding of ICG and provided sufficient contrast. At the same concentration, intense fluorescence with C-albumin-ICG was observed in the damaged areas (FIG. 4). By histological examination with Toluidine blue, the intensely fluorescent area corresponded to an area where the surface was damaged.

### Effects of Cationic Albumin on Chondrocyte Proliferation

We elucidated the molecular mechanism of effects of cationic albumin on chondrocytes proliferation using a cartilage organoid model. Cartilage organoids were formed with isolated chondrocytes, which accumulated newly synthesized cartilage extracellular matrix within the spherical organoids.

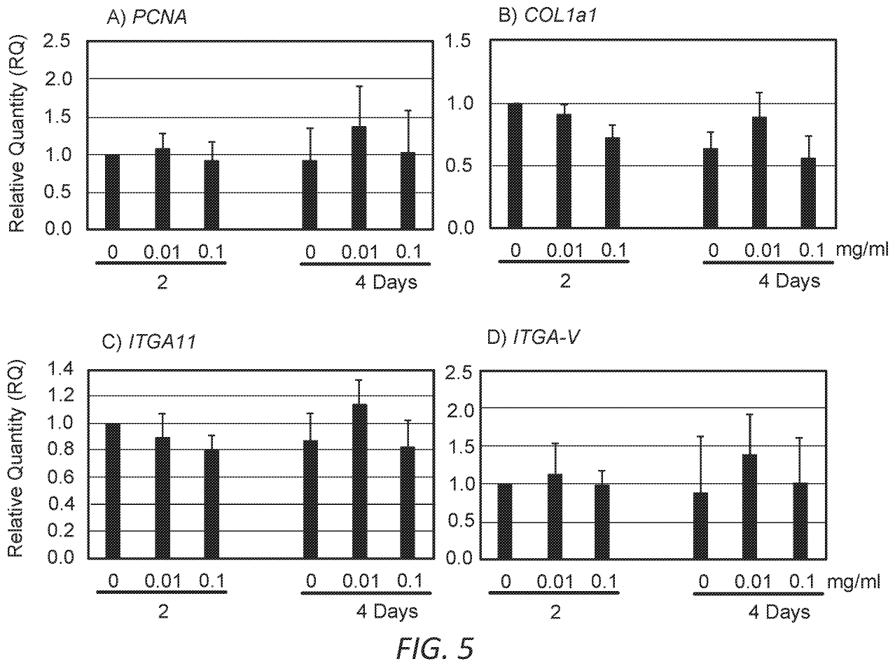

With cationic albumin at 0.01 mg/ml, human articular chondrocytes showed trends of upregulation of PCNA, COL1α1, ITGA-V, ITGA11 compared to no cationic albumin (0 mg/ml) at day 4 (FIG. 5).

The results showed that human cationic albumin stimulated chondrocyte proliferation due to upregulation of PCNA. Major extracellular matrix, collagen type-1 was upregulated. Simultaneously, an adhesion molecule, integrin-11, binding to collagen type-1 and an adhesion molecule, integrin-V binding to fibronectin were also upregulated. Thus, cationic albumin stimulates chondrocytes to produce extracellular adhesion sites, gains adhesion molecules in a cell membrane, and to proliferate.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating an area of damage to a surface of articular cartilage, the method comprising contacting the damaged surface with an effective amount of cationized-albumin (C-albumin).

2. (canceled)

3. The method of claim 1, wherein the C-albumin has a net charge of 23±3.

4. The method of claim 1, wherein the C-albumin is in a composition further comprising a hydrogel that increases viscosity.

5. The method of claim 4, wherein the hydrogel comprises one or more of collagen, gelatin, alginate, hyaluronic acid, heparin, chondroitin sulfate, chitosan, poly(ethylene glycol) (PEG), or poly(vinyl alcohol).

6. A method of identifying an area of damage to a surface of articular cartilage, the method comprising:
contacting the surface with a sufficient amount of cationized-albumin (C-albumin) conjugated to a near-infrared (NIR) imaging moiety;
irradiating the surface with near-infrared light from a source;
detecting an area of fluorescence emitted by the NIR imaging moiety, and
identifying the area of fluorescence as a damaged area.

7. The method of claim 6, wherein the near-infrared imaging moiety is indocyanine green (ICG), and the method comprises irradiating the surface with light between 600 nm and 900 nm, and detecting emitted fluorescence between 750 nm and 950 nm.

8. The method of claim 6, comprising using an endoscope that emits near-infrared light and detects fluorescence emitted by the NIR imaging moiety.
